(19) United States
(12) Patent Application Publication
(10) Pub. No.: US 2014/0234317 A1
(43) Pub. Date: Aug. 21, 2014

(54) METHODS FOR PREVENTING TOXIC DRUG-DRUG INTERACTIONS IN COMBINATION THERAPIES COMPRISING ANTI-ERBB3 AGENTS

(71) Applicants: Matthew David Onsum, San Francisco, CA (US); Clet Niyikiza, Gulph Mills, PA (US); Victor Moyo, Ringoes, NJ (US); William Kubasek, Belmont, MA (US); Akos Czibere, Boston, MA (US)

(72) Inventors: Matthew David Onsum, San Francisco, CA (US); Clet Niyikiza, Gulph Mills, PA (US); Victor Moyo, Ringoes, NJ (US); William Kubasek, Belmont, MA (US); Akos Czibere, Boston, MA (US)

(73) Assignee: Merrimack Pharmaceuticals, Inc., Cambridge, MA (US)

(21) Appl. No.: 14/116,061

(22) PCT Filed: May 4, 2012
(86) PCT No.: PCT/US2012/036619

§ 371 Date: Apr. 30, 2014

Related U.S. Application Data
(60) Provisional application No. 61/483,195, filed on May 6, 2011.

(51) Int. Cl. C07K 16/32; A61K 39/395; A61K 31/517; A61K 31/5377

(52) U.S. Cl. CPC C07K 16/32; A61K 31/5377; A61K 39/39558; A61K 31/517; A61K 2039/505
USPC 424/136.1; 544/293; 544/119; 530/387.7; 514/234.5; 424/138.1; 514/266.4

(57) ABSTRACT

Methods are disclosed for preventing toxic drug-drug interactions during combination cancer therapy with a drug that is an anti-ErbB3 agent, such as an anti-ErbB3 antibody, together with a drug that is a tyrosine kinase inhibitor and/or a drug that binds to alpha-1 acid glycoprotein (e.g., erlotinib). Health care practitioners obtaining any one of the drugs are warned that when co-administering the drug that is an anti-ErbB3 agent with either or both of a drug that is a tyrosine kinase inhibitor and a drug that binds to alpha-1 acid glycoprotein, at least one of the co-administered drugs should be administered using a reduced dosage to prevent toxicity. In a reduced dosage, the amount of drug administered per unit time is reduced as compared to a dose that would be administered if the drug was administered as monotherapy. The reduced dosage can be, for example, a reduced drug dose or a reduced drug dosing frequency, or both. Compositions useful in practicing the disclosed methods are also provided.

### RELATED APPLICATIONS

This application claims priority to U.S. Provisional Application No. 61/483,195 (filed May 6, 2011), which is incorporated by reference, in its entirety, for any and all purposes.

### BACKGROUND

Excessive signaling activity mediated by cell surface ErbB/HER family receptors, e.g., due to receptor overexpression, is a characteristic of many types of tumor cells. Such excessive signaling is understood to promote expression of malignant cellular phenotypes. This understanding has allowed for the development of therapeutic treatments that treat cancers by targeting and reducing the signaling activity mediated by such receptors. For example, tyrosine kinase inhibitors (TKIs) such as erlotinib (e.g., erlotinib hydrochloride, Tarceva®) and gefitinib (Tressa®) specifically inhibit the mitogenic tyrosine kinase activity of certain ErbB/HER receptors expressing such activity (ErbB3 does not express tyrosine kinase activity) and are thereby useful for the treatment of various cancers.

The approach of administering a single drug to treat a disease or disorder (e.g., a therapeutic monoclonal antibody to treat a cancer) in the absence of administration of other therapeutic agents that are administered to treat the same disease or disorder is referred to as monotherapy. In the treatment of cancers, the co-administration of pluralities of anti-cancer drugs (combination therapy) often provides better treatment outcomes than monotherapy. Many drugs are known to alter the bioavailability of other drugs when both drugs are co-administered. For example, certain drugs can alter the degree of plasma protein binding of other drugs. If the amount of a first drug in a patient's bloodstream that is bound to a plasma protein such as serum albumin or alpha 1-acid glycoprotein (AAG) is altered (e.g., by combination therapy with a second drug), the bioavailability in the bloodstream of the first drug is thereby also altered.

When a first drug increases the bioavailability of a second drug, the resulting increased exposure to the second drug can be toxic. As new drugs are first used in combination therapies, unforeseen, hazardously toxic drug-drug interactions may be observed. It is important for clinicians to avoid causing such drug-drug interactions (DDIs), as the resulting toxicities can result in significant morbidity and mortality.

Anti-ErbB3 agents (e.g., antibodies) constitute a novel class of anti-cancer drugs, at least three of which are currently undergoing clinical trials in human cancer patients.

Thus approaches for safely administering novel combination therapies for cancer treatment comprising administration of anti-ErbB3 agents are needed. Such approaches include compositions and methods to prevent or reduce toxicities resulting from such combination therapies and to provide other benefits.

### SUMMARY

Provided are methods of reducing the risk of harmful drug-drug interactions when supplying or administering drugs for combination therapy methods. These methods involve combinations of an anti-ErbB3 agent and an additional therapeutic agent that is not an anti-ErbB3 agent. In one embodiment, the additional therapeutic agent is either a tyrosine kinase inhibitor (TKI) or a drug that binds to the plasma protein alpha 1-acid glycoprotein with a sufficient binding affinity that such binding can alter the bioavailability of the drug in the bloodstream of a patient (an AAG binder—hereinafter an “AAGB”). AAG binding drugs are also described in U.S. Pat. No. 5,750,493. Many TKIs are AAGBs. Thus erlotinib and gefitinib are (non-limiting) examples of both TKIs and of AAGBs. Other examples of TKIs that are known to be AAGBs include imatinib and lapatinib.

As set forth in the Examples below, it is been observed that administration of an anti-ErbB3 agent to patients who receive co-administration of a TKI that is an AAGB (at the manufacturer's recommended dosage) increases the frequency of observation of signs and/or symptoms of toxicity associated with the TKI as compared to the frequency of such signs and/or symptoms observed in patients who receive the TKI as monotherapy at the same dosage. It is additionally observed that increasing the dosage of the anti-ErbB3 agent further increases the frequency of observation of signs and/or symptoms of toxicity associated with the TKI in such patients. It is further observed that the plasma levels of the TKI in many of the patients exhibiting such signs and/or symptoms of TKI toxicity are higher than the plasma levels of patients not exhibiting such signs and symptoms.

These observations indicate that co-administration of an anti-ErbB3 agent and a drug that is a TKI and/or an AAGB results in a drug-drug interaction that can produce signs and symptoms of toxicity. In one aspect it is believed that the plasma concentration of free AAGB (unbound to plasma proteins) is increased by the co-administration of an AAGB and an anti-ErbB3 antibody. In another aspect it is believed that total plasma concentration of a TKI (e.g., the AUC) is increased by the co-administration of a TKI with an anti-ErbB3 antibody.

Accordingly, methods and compositions are provided herein that allow for the co-administration of such drug combinations while reducing the risk of harmful drug-drug interactions.

Supplying methods disclosed herein include:

Supplying a TKI or an AAGB to a drug distributor, the TKI or AAGB being supplied in a container comprising 1) the TKI or AAGB formulated for administration to a patient, and 2) a recorded or printed warning a medical professional; which warning indicates that when the TKI or AAGB is co-administered to the patient with an ErbB3 inhibitor, a dose reduction of the TKI or AAGB should be considered.

Supplying an ErbB3 inhibitor to a drug distributor, the ErbB3 inhibitor being supplied in a container comprising 1) the ErbB3 inhibitor formulated for administration to a patient, and 2) a recorded or printed warning a medical professional; which warning indicates that when the ErbB3 inhibitor is co-administered to the patient with a TKI or an AAGB, a dose reduction of the TKI or AAGB should be considered.

Supplying a TKI to a drug distributor, the TKI being supplied in a container comprising 1) the TKI formulated for administration to a patient, and 2) a recorded or printed warning a medical professional; which warning indicates that when the TKI is co-administered to the patient with an ErbB3 inhibitor, a dose reduction of the TKI should be considered.

Supplying a drug that is an AAGB to a drug distributor, the AAGB being supplied in a container comprising 1) the AAGB formulated for administration to a patient, and 2) a recorded or printed warning a medical professional; which warning indicates that when the AAGB is co-administered to the patient with an ErbB3 inhibitor, a dose reduction of the AAGB should be considered.

In certain embodiments of each of the warnings in the 4 preceding paragraphs, the warning indicates that the reduction for a patient who is a cigarette smoker should be a reduction of a lesser magnitude than the reduction for a patient who is not a cigarette smoker.

Supplying an ErbB3 inhibitor to a drug distributor, the ErbB3 inhibitor being supplied in a container comprising 1) the ErbB3 inhibitor formulated for administration to a patient, and 2) a recorded or printed warning a medical professional; which warning indicates that when the ErbB3 inhibitor is co-administered to the patient with a drug that is an AAGB, a dose reduction of the AAGB should be considered, or the warning indicates that when the ErbB3 inhibitor is co-administered to the patient with a drug that is an AAGB, a dose reduction of the ErbB3 inhibitor should be considered.

In preferred embodiments, the a recorded or printed warning comprises one or more of a recorded audio warning, a recorded video warning, a warning recorded in computer readable form, or printed words, printed pictures, printed bar code(s), printed QR (quick response) code(s), or the like.

In other embodiments: the medical professional is a physician, a physician's assistant, a nurse, a pharmacist, or a pharmacy technician; the TKI or AAGB is erlotinib or gefitinib and the warning further indicates that a dosage reduction in 50 mg increments or 25 mg increments is suggested; the TKI or AAGB is erlotinib or gefitinib and the warning further indicates that a dosage reduction in 125 mg increments or about 62 mg increments is suggested; the TKI or AAGB is gefitinib and the warning further indicates that a dosage reduction is suggested consisting of administering the drug every other day rather than every day; the patient is suffering from a cancer for which treatment with either or both of an AAGB or a TKI is indicated; and the patient is suffering from a cancer for which treatment with an ErbB3 inhibitor is indicated and the ErbB3 inhibitor is an anti-ErbB3 antibody (such as any of those disclosed below, including but not limited to MM-121 or AMG888).

Therefore, in one aspect, a method of treatment is provided for preventing a toxic DDI resulting from combination therapy treatment of a cancer patient with an anti-ErbB3 agent and a TKI or AAGB wherein a first TKI or AAGB dosage has been recommended by a manufacturer of the TKI or AAGB for administration of the TKI or AAGB in the absence of co-administered anti-ErbB3 agent and a second TKI or AAGB dosage has been recommended or suggested for consideration by the manufacturer or distributor for administration of the TKI or AAGB in the presence of co-administered anti-ErbB3 agent, which second dosage is a reduced dosage that provides a reduced dosage of the TKI or AAGB as compared to the first dosage; the method comprising co-administering the TKI and the anti-ErbB3 agent to the cancer patient, with the TKI administered according to the reduced dosage.

In another aspect, a method is provided for combination therapy, the therapy being for treatment of a patient suffering from a cancer, the combination therapy being administration of both of an anti-ErbB3 drug and either a tyrosine kinase inhibitor (TKI) drug or an alpha 1 acid glycoprotein binding (AAGB) drug, the method comprising administering both the anti-ErbB3 drug and the TKI drug or the AAGB drug to the patient, wherein either or both of 1) the TKI drug or the AAGB drug and 2) the anti-ErbB3 drug is administered to the patient in either or both of a modified dose or a modified dosing frequency.

In various aspects, each modified dose of a drug may be a reduced dose as compared to the dose recommended for monotherapy treatment with the drug in such a patient (i.e., the monotherapy dose). In various embodiments, the TKI drug or the AAGB drug is administered at the reduced dose and the anti-ErbB3 drug is not administered at the reduced dose; the anti-ErbB3 drug is administered at the reduced dose and the TKI drug or the AAGB drug is not administered at the reduced dose; each of 1) the TKI drug or the AAGB drug, and 2) the anti-ErbB3 drug, are administered at a reduced dose; the amount of each dose is reduced by 10-75%; the frequency of dosing of at least one drug is reduced as compared to the recommended frequency of dosing for the at least one drug; the TKI drug or the AAGB drug is administered at a reduced frequency and the anti-ErbB3 drug is not administered at a reduced frequency; the anti-ErbB3 drug is administered at a reduced frequency and the TKI drug or the AAGB drug is not administered at a reduced frequency; each of 1) the TKI drug or the AAGB drug, and 2) the anti-ErbB3 drug are administered at a reduced frequency; each reduced frequency is obtained by extending intervals between administrations by at least one day; and

the anti-ErbB3 drug exhibits a first serum half life;

the TKI drug or the AAGB drug exhibits a second serum half life; and

administration of the anti-ErbB3 drug to the patient occurs within 1 to 3 first serum half lives before administering the TKI drug or the AAGB drug; or

administration of the anti-ErbB3 drug occurs within 1 to 3 second serum half lives after administering the TKI drug or the AAGB drug.

In one embodiment of each of the methods disclosed above, the reduced dosage comprises reducing the amount of AAGB or TKI administered in each dose (i.e., each time the AAGB or TKI is administered). In this embodiment, each dose can be reduced by 1/10, ⅛, ⅙, ⅕, ¼, ⅓, ½, ⅔ or ¾, or by about 10, 25, 50, 100, 150, 200, 250, 300, 400, 425, 500, or 750 mg, as compared to each dose of the first dosage. In another embodiment of each of the methods disclosed above, the reduced dosage comprises reducing the frequency of administration of doses of the AAGB or TKI. In a further embodiment of each of the methods disclosed above, the reduced dosage comprises reducing both the amount of each dose and the frequency of administration of the AAGB or TKI.

In non-limiting embodiments of each of the methods disclosed above, the anti-ErbB3 agent inhibits the phosphorylation of ErbB3 when ErbB3 is contacted with heregulin (e.g., in vitro). In other non-limiting embodiments the anti-ErbB3 agent is a TKI, e.g., lapatinib. In other non-limiting embodiments the anti-ErbB3 agent is a monospecific anti-ErbB3 antibody. In one such embodiment, the anti-ErbB3 agent is monoclonal anti-ErbB3 antibody MM-121, as described (as “Ab #6”) in U.S. Pat. No. 7,846,440, or is an antibody that competes with MM-121 for binding to ErbB3. In another such embodiment, the anti-ErbB3 agent is any of the other anti-ErbB3 antibodies described in U.S. Pat. No. 7,846,440, such as Ab #3, Ab #14, Ab #17 or Ab #19 or is an antibody that competes with Ab #3, Ab #14, Ab #17 or Ab #19 for binding to ErbB3. Additional examples of anti-ErbB3 antibodies that may be administered in accordance with the methods disclosed herein include antibodies disclosed in U.S. patents and patent publications Nos.; U.S. Pat. No. 7,285,649; 20100310557; and 20100255010, as well as antibodies 1B4C3 and 2D1D12 (U3 Pharma AG), both of which are described in, e.g., U.S. Publication No. 20040197332, anti-ErbB3 antibodies disclosed in U.S. Pat. No. 7,705,130 including but not limited to the anti-ErbB3 antibody referred to as AMG888 (U3-1287-U3 Pharma AG and Amgen) and the monoclonal antibodies (including humanized versions thereof), such as 8B8, described in U.S. Pat. No. 5,968,511. Other such examples include anti-ErbB3 antibodies that are multi-specific antibodies and comprise an anti-ErbB3 antibody linked to at least a second antibody. For example, such a multi-specific antibody may be a bispecific antibody, e.g. one comprising an anti-ErbB3 antibody linked to a second antibody, such as an anti-IGFR1 antibody. In one embodiment, the bispecific antibody comprises an anti-ErbB3 antibody linked to an anti-ErbB2 antibody, as described (e.g., as “B2B3-1” or B2B3-2) in International Patent Application No.: PCT/US2009/040259 (published as WO 2009/126920). Other suitable bispecific anti-ErbB3 agents are also disclosed therein. A monospecific anti-ErbB3 antibody may be administered at a therapeutically effective dosage, e.g., a monotherapy dose.

In other embodiments of the above combination methods, the TKI or AAGB is erlotinib or gefitinib, the AAGB is a basic compound, the AAGB is an anti-cancer drug, the monotherapy dose for erlotinib is 150 mg/day, and the reduced dose for erlotinib is 100 mg/day; or the monotherapy dose for erlotinib is 150 mg/day, and the reduced dose for erlotinib is 125 mg/day; or the monotherapy dose for erlotinib is 150 mg/day and the reduced dose for erlotinib is 75 mg/day; or the monotherapy dose for erlotinib is 150 mg/day and the reduced dose for erlotinib is 50 mg/day; or the monotherapy dose for erlotinib is 150 mg/day and the reduced dose for erlotinib is 25 mg/day; or the monotherapy dose for erlotinib is 100 mg/day, and the reduced dose for erlotinib is 75 mg/day or the monotherapy dose for erlotinib is 100 mg/day and the reduced dose for erlotinib is 50 mg/day; the monotherapy dose for erlotinib is 100 mg/day, and the reduced dose for erlotinib is 25 mg/day; or the monotherapy dose for gefitinib is 250 mg/day, and the reduced dose for gefitinib is 150 mg/day or 125 mg/day or 100 mg/day or about 62 mg/day or 50 mg/day; or the monotherapy dose for gefitinib is 250 mg/day, and the reduced dose for gefitinib is 250 mg/every other day.

In yet another embodiment of the above combination methods, the reduced dose for the anti-ErbB3 antibody is about ½ or about ¼ of the monotherapy dose of the anti-ErbB3 antibody.

The methods disclosed above can be used in regard to the treatment of any cancer susceptible to treatment by an anti-ErbB3 agent. Non-limiting examples of types of cancers to be treated include cancers of the breast, ovary, kidney, lung, prostate, pro static intraepithelial neoplasia, head and neck, brain, spinal cord, liver, bone, skin (e.g., melanoma), spleen, testicle, bladder, and thyroid, gastrointestinal system (e.g., colon, rectum, pancreas, gall bladder, stomach, and esophagus, along with colorectal cancer and oral/pharyngeal cancer), as well as sarcomas such as clear cell sarcoma or Kaposi's sarcoma.

For any of the above methods, for the anti-ErbB3 agent and the TKI or AAGB may be administered simultaneously. Alternatively, the anti-ErbB3 agent may be administered initially, followed by the TKI or AAGB, or the TKI or AAGB may be administered initially, followed by the anti-ErbB3 agent. The two drugs are administered within a time period sufficient so that the patient is exposed for a time to the therapeutic effectiveness of both agents at the same time. For example, in one embodiment, the TKI or AAGB are administered within 1-3 weeks after administration of the anti-ErbB3 agent. In another embodiment, the anti-ErbB3 agent is administered within 2-3 days after administration of the TKI or AAGB. In other embodiments of the above methods, the anti-ErbB3 agent exhibits a first serum half life, the TKI or AAGB exhibits a second serum half life, and administration of the anti-ErbB3 agent to the patient occurs within 1, 2 or 3 first serum half lives before administering the TKI or AAGB, or administration of the anti-ErbB3 agent occurs within 1, 2, or 3 second serum half lives after administering the TKI or AAGB.

For any of the above methods, wherein the patient is a cigarette smoker, and wherein the patient is given a reduced dose of a TKI drug or ErbB3 inhibitor, the reduction is less than the reduced dose of the TKI drug or ErbB3 inhibitor that would be given to the same patient if the patient were a non-smoker.

In one embodiment, the AAGB is a TKI such as erlotinib or gefitinib. In another embodiment, the AAGB is a basic drug or a neutral lipophilic drug or an anti-cancer agent.

In another aspect, packaged formulations for treating cancer are provided. The packaged formulations comprise a drug in a container, wherein the drug is a TKI or an AAGB or an anti-ErbB3 agent (e.g., an ErbB3 inhibitor such as an anti-ErbB3 antibody) formulated for administration to a patient (e.g., formulated in a pharmaceutically acceptable carrier) and the package further comprises a recorded or printed warning a medical professional or a patient as described above. For example, the drug can be a TKI and the warning can indicate that when the TKI is co-administered with an ErbB3 inhibitor, a dose modification of the TKI should be considered; the drug can be an ErbB3 inhibitor and the warning can indicate that when the ErbB3 inhibitor is co-administered with a TKI, a dose modification of the TKI should be considered; or the drug can be an ErbB3 inhibitor and the warning can indicate that when the ErbB3 inhibitor is co-administered with a TKI, a dose modification of the ErbB3 inhibitor should be considered. Preferably, the dose modification is a dose reduction (as compared to the dose used for monotherapy). In other embodiments, the drug can be a TKI and the warning can indicate that when the TKI is co-administered with an ErbB3 inhibitor, the TKI should be administered at a modified dose; the drug can be an ErbB3 inhibitor and the warning can indicate that when the ErbB3 inhibitor is co-administered with a TKI, the TKI should be administered at a modified dose; or the drug can be an ErbB3 inhibitor and the warning can indicate that when the ErbB3 inhibitor is co-administered with a TKI, the ErbB3 inhibitor should be administered at a modified dose. Preferably, the modified dose is a reduced dose (as compared to the dose used for monotherapy).

In another aspect, packaged formulations for treating cancer are provided. The packaged formulations comprise a TKI or an AAGB or an anti-ErbB3 drug (e.g., an anti-ErbB3 antibody) in a pharmaceutically acceptable carrier and the package further comprises instructions for use in accordance with the combination methods (of co-administering an ant-ErbB3 agent with either a TKI or an AAGB) described above.

### DETAILED DESCRIPTION

Herein provided are methods of combination therapy for treating cancer in a patient. In these methods, the cancer patient is treated with both an anti-ErbB3 agent and erlotinib or another AAGB. In these combination therapy methods, the dosage for the erlotinib or other AAGB is reduced as compared to a therapeutically effective dosage when the erlotinib or other AAGB is used alone.

These methods are based, at least in part, on the discovery that when an anti-ErbB3 agent (e.g., an anti-ErbB3 antibody) is co-administered with erlotinib, the plasma concentration of the erlotinib increases when the concentration of the anti-ErbB3 agent increases (see Example 1).

### DEFINITIONS

The terms “combination therapy”, “co-administration” and “co-administered” refer to the administration of at least two therapeutic agents to a patient either simultaneously or within a time period during which the effects of the first administered therapeutic agent are still operative in the patient when the second administered therapeutic agent is administered.

The term “monotherapy” refers to administering a single drug to treat a disease or disorder in the absence of co-administration of other therapeutic agents that are being administered to treat the same disease or disorder.

The terms “therapeutic agent” or “drug” are intended to encompass any and all compounds that have an ability to decrease or inhibit the severity of the symptoms of a disease or disorder, or increase the frequency and/or duration of symptom-free or symptom-reduced periods in a disease or disorder, or inhibit or prevent impairment or disability due to a disease or disorder affliction, or inhibit or delay progression of a disease or disorder, or inhibit or delay onset of a disease or disorder, or inhibit or prevent infection in an infectious disease or disorder. Non-limiting examples of therapeutic agents include small (i.e., less than about 700 Daltons) organic molecules, monoclonal antibodies, bispecific antibodies, recombinantly engineered biologics, RNAi compounds and the like.

An “AAGB” is a drug that exhibits sufficient binding affinity for the human plasma protein alpha 1-acid glycoprotein (AAG) so that such binding can alter the bioavailability of the drug in a patient. The term “alpha 1-acid glycoprotein” (also referred to as AAG, AGP or orosomucoid) is intended to encompass all genetic variants of this plasma protein, including the A variant and the F1/S variant. Variants of AAG, and drug binding thereto, have been described in the art.

A TKI is a small molecule (i.e., less than about 700 Daltons) tyrosine kinase inhibitor. TKIs include, e.g., afatinib, axitinib, bosutinib, canertinib, cediranib, crizotinib, damnacanthal, dasatinib, erlotinib, gefitinib, imatinib, lapatinib, lestaurtinib, neratinib, nilotinib, pazopinib, pelitinib, regorafenib, ruxolitinib, semaxanib, sorafenib, sunitinib, toceranib, tofacitinib, UCN-01, vandetanib, and vatalinib.

The term “anti-ErbB3 agent” refers to any therapeutic agent that binds to ErbB3 or binds to an ErbB3-specific ligand or blocks the expression of ErbB3, and thereby inhibits the activity of cellular signaling mediated by ErbB3. Non-limiting examples of types of anti-ErbB3 agents include antibodies, bispecific antibodies, ligand analogs, soluble forms of ErbB3 or the ErbB3 ectodomain, ErbB3 specific RNAi molecules, and similar biologic agents, as well as certain tyrosine kinase inhibitors.

The terms “anti-ErbB3 agent”, “anti-ErbB3 drug” and “ErbB3 inhibitor” are used interchangeably herein.

The term “antibody” includes whole antibodies and any antigen binding fragment (i.e., “antigen-binding portion,” e.g., Fabs) or single chains thereof (e.g., scFvs) as well as bispecific antibodies and similar engineered variants, provided that they retain the binding specificity of an antibody.

An “anti-ErbB3 antibody” is an antibody that immunospecifically binds to the ectodomain of ErbB3. Such binding to ErbB3 typically exhibits a K_{d} of 50 nM or less (i.e., a binding affinity corresponding to a K_{d} value of 50 nM, or a higher binding affinity as indicated by a lower K_{d} value) e.g., as measured by a surface plasmon resonance assay or a cell binding assay. In one aspect the anti-ErbB3 antibodies inhibit EGF-like ligand-mediated phosphorylation of ErbB3 and/or inhibit ErbB2/ErbB3 complex formation in living cells. EGF-like ligands include any of the forms of each of the following: heregulin, EGF, TGFα, betacellulin, heparin-binding epidermal growth factor, biregulin, epigen, epiregulin, and amphiregulin.

The term “bispecific” as used herein refers to a protein comprising two antigen-binding sites, a first binding site exhibiting immunospecific binding to a first antigen or epitope and a second binding site exhibiting immunospecific binding to a second antigen or epitope distinct from the first.

The term “dosage” refers to parameters for administering a drug in fixed quantities per unit time (e.g., per hour, per day, per week, per month, etc.) to a subject, such parameters including size of each dose (which may be administered as a unit; e.g., taken at once orally or injected as a single bolus; or continuously; e.g., as an intravenous infusion over a period of minutes or hours) and frequency of administration of separate doses.

The term “dose” refers to an amount of drug given in a single administration.

The term “therapeutically effective dosage” refers to a dosage that has been shown to successfully achieve a desired therapeutic result or effect. For example, a therapeutically effective dosage can be a dosage that is recommended for use of an agent in monotherapy (administration of the agent alone, not in combination with one or more additional agents) to achieve the desired therapeutic result or effect.

The term “reduced dosage” refers to a dosage in which one or more parameters of the dosage have been reduced (e.g., the size of at least one dose has been lowered or the frequency of administration has been diminished) as compared to another dosage (e.g., as compared to a therapeutically effective dosage for monotherapy).

The terms “treat,” “treating,” and “treatment,” refer to therapeutic or preventative measures described herein. Methods of “treatment” employ administration, to a subject, for example, a subject having cancer, of one or more drugs, in order to prevent, cure, delay, reduce the severity of, or ameliorate one or more symptoms of cancer in the subject, e.g., to inhibit the growth or division of cancer cells or to inhibit movement (taxis), metastasis, or invasiveness of cancer cells in the subject.

The term “recommended” generally means as advised by the manufacturer. In particular, recommended refers to the teachings of any of the dose and dosage frequency instructions and suggestions comprised by the prescribing information for the drug in question.

Additional aspects regarding the foregoing and various additional aspects of this disclosure are described in further detail in the following subsections, which should not be construed as limiting.

Along with many other drugs, certain TKIs are known to bind to plasma alpha 1-acid glycoprotein (AAG). Such binding reduces drug bioavailability by reducing the amount of free drug in the blood that can interact with (e.g., enter) cells. All TKIs in clinical use (e.g., erlotinib, dasatinib, afatinib, gefitinib, imatinib, pazopinib, lapatinib, sunitinib, nilotinib and sorafenib) exhibit high plasma protein binding, and at least erlotinib, lapatinib, imatinib, and gefitinib have been reported to exhibit significant binding to AAG that can alter their bioavailability, and are thus AAGBs.

### I. COMBINATION THERAPY

The ability of the anti-ErbB3 agent to alter the pharmacokinetics of a co-administered AAGB or TKI can be evaluated by measuring serum drug levels and calculating pharmacokinetic parameters. Pharmacokinetic parameters referred to herein (e.g., Example 1) describe the in vivo characteristics of a drug over time. These include plasma concentration (C), as well as Cₘₐₓ, Tₘₐₓ, and AUC. The term “Cₘₐₓ” refers to the measured plasma concentration of the active agent at the point of maximum, or peak, concentration following an administration. The term “Tₘₐₓ” refers to the time from drug administration until Cₘₐₓ is reached. The term “AUC” refers to the area under the curve of a graph of the measured plasma concentration of an active agent vs. time, measured from one time point to another time point. For example AUC₀₋ₜ is the area under the curve of plasma concentration versus time from time 0 to time t, where time 0 is the time of initial administration of the drug. Time t can be the last time point with measurable plasma concentration for an individual drug.

Given that administration of an anti-ErbB3 agent alters the AUC of a co-administered AAGB or TKI, in the disclosed methods the dosage of the AAGB is reduced, as compared to a dosage that would be used when the AAGB is administered without co-administration of the anti-ErbB3 agent. Accordingly, in one aspect, a method is provided for combination therapy for treatment of a cancer patient with an anti-ErbB3 agent and a drug that binds to alpha 1-acid glycoprotein (AAG), the method comprising:

a) administering the anti-ErbB3 agent;

b) determining a monotherapy dosage for administration of the AAGB;

c) determining a co-administration dosage for administration the AAGB when co-administered with the anti-ErbB3 agent, which co-administration dosage is a reduced dosage as compared to the monotherapy dosage; and

d) co-administering the anti-ErbB3 agent and the AAGB, with the AAGB being administered according to the co-administration dosage, to the cancer patient.

In one embodiment, the co-administration dosage comprises reducing the dosage of the AAGB. That is, the reduction comprises reducing the size of at least one of the doses of the AAGB. In various embodiments, the dosage is reduced by 10-75%, 20-60%, or 10-50% as compared to the dosage regimen that would be administered when the AAGB is administered in the absence of co-administration of the anti-ErbB3 agent (e.g., as monotherapy).

In another embodiment, the reduced dosage comprises reducing the frequency of dosing. That is, the reduction comprises reducing the number of doses that are administered per unit time (e.g., per day, per every two days, per week). Non-limiting examples of reducing the frequency of dosing for combination therapy include administering an agent every two days or every three days as compared to daily monotherapy administration, or administering the agent once a day as compared to twice or thrice daily monotherapy administration, or administering the agent once a week as compared to twice a week monotherapy administration.

In yet another embodiment, the reduced dosage comprises reducing both the dosage and the frequency of dosing. That is, the reduction can comprise reducing both the size of the dose and the number of doses administered per unit time.

In yet another embodiment, the timing of co-administration of the two agents is determined based on the serum half-lives of the two agents. For example, in one embodiment, the anti-ErbB3 agent exhibits a first serum half life, the AAGB exhibits a second serum half life, and administration of the anti-ErbB3 agent to the patient occurs within 1 to 3 first serum half lives before administering the AAGB, or administration of the anti-ErbB3 agent occurs within 1 to 3 second serum half lives after administering the AAGB.

With respect to the timing of co-administration of the anti-ErbB3 agent and the AAGB, the key factor is that the in vivo activity (e.g., plasma concentration) of the anti-ErbB3 agent is still sufficiently high that it affects the pharmacokinetics of the co-administered AAGB such that the dosage for one or both of the agents is adjusted downward to compensate for this effect on the pharmacokinetics of the AAGB.

Example 1 provides non-limiting exemplary dosages for an anti-ErbB3 agent (anti-ErbB3 antibody) of 6 mg/kg and 12 mg/kg, administered intravenously once per week. Thus, in one embodiment, the monotherapy dose is 12 mg/kg/week and the reduced dose is ½ or ¼ of the monotherapy dose, i.e., the reduced dose is 6 mg/kg/week or 3 mg/kg/week; in another aspect, the monotherapy dose is 6 mg/kg/week and the reduced dose is ½ or ¼ of the monotherapy dose, i.e., the reduced dose is 3 mg/kg/week or 1.5 mg/kg/week. In another aspect the monotherapy dose is 12 mg/kg/week or 6 mg/kg/week and the reduced dose is ½ of the monotherapy dose or the reduced dose is ¼ of the monotherapy dose, i.e., the reduced dose is 12 mg/kg/every two weeks or 6 mg/kg/every two weeks or the reduced dose is 12 mg/kg/every four weeks or 6 mg/kg/every four weeks. Examples 2, 3 and 4 provide exemplary combination dosage regimens for an anti-ErbB3 antibody combined with a TKI or AAGB, such as erlotinib or gefitinib. In one embodiment, the anti-ErbB3 antibody is used at a monotherapy dose and the dose for the TKI or AAGB is reduced from its monotherapy dose. For example, the monotherapy dose of erlotinib of 150 mg/day is reduced to 100 mg/day when used in combination with the anti-ErbB3 antibody, or the monotherapy dose of gefitinib of 250 mg/day is reduced, when used in combination with the anti-ErbB3 antibody, to 125 mg/day, or the monotherapy dose of gefitinib of 250 mg/day is reduced, when used in combination with the anti-ErbB3 antibody, to 250 mg/kg/48 hrs. In another embodiment, the recommended monotherapy dose of the TKI, such as 150 mg/day for erlotinib or 250 mg/day for gefitinib, is maintained in the combination dosage regimen and the dose of the anti-ErbB3 antibody is reduced from its monotherapy dose. For example, the monotherapy dose of the anti-ErbB3 antibody can be reduced by e.g., 10-75%, for example, by 10%, 20%, 25%, 30%, 40%, 50%, 60%, 70% or 75% as compared to the monotherapy dose so that, for example, when the monotherapy dose of the anti-ErbB3 antibody is 12 mg/kg/day, the reduced dose can be about 11 mg/kg/day, about 9.5 mg/kg/day, 9 mg/kg/day, about 8.5 mg/kg/day, about 7 mg/kg/day, 6 mg/kg/day, about 5 mg/kg/day, about 3.5 mg/kg/day or 3 mg/kg/day.

Other suitable reduced dosages will be readily apparent to the ordinarily skilled artisan based on the guidance provided herein and the monotherapy doses for anti-ErbB3 agents (e.g., anti-ErbB3 antibodies) and the monotherapy doses for drugs that bind to AAG (e.g., erlotinib or gefitinib).

In certain embodiments, further dose adjustments are made if the patient is a cigarette smoker. In particular, a TKI dose is selected for use in combination therapy with an anti-ErbB3 agent in a smoker that is higher than the dose that would be selected for use in combination therapy with an anti-ErbB3 agent for a patient who is a non-smoker. For example, a 50 mg/day dose of erlotinib for use in combination therapy with an anti-ErbB3 agent in a non-smoker would be increased, e.g., to 75 or 100 or 125 or 150 mg/kg in a patient who smokes cigarettes. In another embodiment, a patient that is a cigarette smoker who is receiving combination therapy of a TKI and an anti-ErbB3 agent is given a higher dose (e.g., about 25%, about 50% about 75% or about 100% higher) of the anti-ErbB3 agent than would be given to a patient who is a non-smoker. In one embodiment, the monotherapy dose of the anti-ErbB3 agent is used in the combination therapy for a patient who is a cigarette smoker, which patient would otherwise, in accordance with the teachings herein, receive a dose of the anti-ErbB3 agent lower than the monotherapy dose.

### II. ANTI-ErbB3 AGENTS

In the combination therapy methods disclosed herein, an anti-ErbB3 agent is administered to the patient. Preferred anti-ErbB3 agents are anti-ErbB3 antibodies, including monoclonal antibodies, recombinant antibodies, human antibodies, humanized antibodies and chimeric antibodies, as well as antigen-binding fragments thereof.

In a preferred embodiment, the anti-ErbB3 antibody comprises MM-121, which is a human anti-ErbB3 antibody currently undergoing Phase II clinical trials. MM-121 and related human anti-ErbB3 antibodies are described in detail in U.S. Pat. No. 7,846,440, U.S. Patent Publication Nos. U.S. 20090291085, U.S. 20100056761, and U.S. 20100266584, and PCT Publication No. WO 2008/100624. In one embodiment, the antibody comprises VH and/or VL regions comprising the amino acid sequences set forth in SEQ ID NOs 1 and 2, respectively. In another embodiment, the antibody comprises CDRH1, CDRH2, and CDRH3 sequences comprising the amino acid sequences set forth in SEQ ID NO: 3 (CDRH1) SEQ ID NO: 4 (CDRH2) and SEQ ID NO: 5 (CDRH3), and/or CDRL1, CDRL2, and CDRL3 sequences comprising the amino acid sequences set forth in SEQ ID NO: 6 (CDRL1) SEQ ID NO: 7 (CDRL2) and SEQ ID NO: 8 (CDRL3). In another embodiment, the antibody comprises heavy and light chains comprising the amino acid sequences set forth in SEQ ID NOs 9 and 10, respectively.

In another embodiment, the antibody comprises VH and/or VL regions comprising the amino acid sequences set forth in SEQ ID NOs 11 and 12, respectively. In another embodiment, the antibody comprises VH and/or VL regions comprising the amino acid sequences set forth in SEQ ID NOs 19 and 20, respectively. In another embodiment, the antibody comprises VH and/or VL regions comprising the amino acid sequences set forth in SEQ ID NOs 27 and 28, respectively. In another embodiment, the antibody comprises VH and/or VL regions comprising the amino acid sequences set forth in SEQ ID NOs 35 and 36, respectively.

Other examples of anti-ErbB3 antibodies and humanized or human versions thereof include the antibodies 1B4C3 (cat #sc-23865, Santa Cruz Biotechnology) and 2D1D12 (U3 Pharma AG), both of which are described in, e.g., U.S. Patent Publication No. 20040197332 and are produced by hybridoma cell lines DSM ACC 2527 or DSM ACC 2517 (deposited at DSMZ), AV-203 (SEQ ID NO:190 (heavy chain) and SEQ ID NO:206 (light chain) in PCT Publication No. WO 2011/136911, Aveo Pharmaceuticals); 8B8 (produced by ATCC® hybridoma #HB-12070™ and described in U.S. Pat. No. 5,968,511 and PCT Publication No. WO 1997/035885); those described in U.S. Pat. No. 7,846,440; the monoclonal antibody Mab 205.10.2 (SEQ ID NO:8 (heavy chain) and SEQ ID NO:10 (light chain) in U.S. Patent Publication No. 20110171222, Roche Glycart); the murine anti-ErbB3 antibody described in U.S. Patent Publication No. 20100310557 (Trellis Biosciences) or a bispecific anti-ErbB3/anti-EGFR antibody (e.g., SEQ ID NO:14 (heavy chain) and SEQ ID NO:13 (light chain) in PCT Publication No. WO 2010/108127, Genentech).

In another embodiment, the anti-ErbB3 antibody is a bispecific antibody (e.g., a fusion protein) comprising an anti-ErbB3 antibody linked to a second antibody. In one embodiment, the bispecific antibody comprises an anti-ErbB3 antibody linked to an anti-ErbB2 antibody. A preferred example of such a bispecific antibody is B2B3-1 as described in PCT/US2009/040259. The antibody components of B2B3-1 are further described in U.S. Pat. No. 7,332,580, as well as in PCT Application PCT/US2006/023479 (published as WO 2007/084181) and PCT Application PCT/US2007/024287 (published as WO 2008/140493).

In another preferred embodiment, the anti-ErbB3 agent is a bispecific antibody comprising an anti-ErbB3 antibody linked to an anti-IGF1R antibody. Such bispecific antibodies are described further in co-pending PCT Application No. PCT/US2010/052712 (published as WO 2011/047180) and co-pending PCT Application No. PCT/US2012/034244.

In other embodiments, the anti-ErbB3 agent is a bispecific antibody comprising a first antibody that specifically binds ErbB3 and a second antibody that specifically binds a protein selected from the group consisting of insulin-like growth factor 2 receptor (IGF2R), insulin-like growth factor (IGF), mesenchymal epithelial transition factor receptor (c-met), hepatocyte growth factor (HGF), epidermal growth factor receptor (EGFR), epidermal growth factor (EGF), heregulin, fibroblast growth factor receptor (FGFR), platelet-derived growth factor receptor (PDGFR), platelet-derived growth factor (PDGF), vascular endothelial growth factor receptor (VEGFR), vascular endothelial growth factor (VEGF), tumor necrosis factor receptor (TNFR), tumor necrosis factor alpha (TNF-α), TNF-β, folate receptor (FOLR), folate, transferrin receptor (TfR), mesothelin, Fc receptor, c-kit receptor, c-kit, α4 integrin, P-selectin, sphingosine-1-phosphate receptor-1 (S1PR), hyaluronate receptor, leukocyte function antigen-1 (LFA-1), CD4, CD11, CD18, CD20, CD25, CD27, CD52, CD70, CD80, CD85, CD95 (Fas receptor), CD106 (vascular cell adhesion molecule 1 (VCAM1), CD166 (activated leukocyte cell adhesion molecule (ALCAM)), CD178 (Fas ligand), CD253 (TNF-related apoptosis-inducing ligand (TRAIL)), ICOS ligand, CCR2, CXCR3, CCR5, CXCL12 (stromal cell-derived factor 1 (SDF-1)), interleukin 1 (IL-1), CTLA-4, receptors alpha and beta, MART-1, gp100, MAGE-1, ephrin (Eph) receptor, mucosal addressin cell adhesion molecule 1 (MAdCAM-1), carcinoembryonic antigen (CEA), Lewis^{Y}, MUC-1, epithelial cell adhesion molecule (EpCAM), cancer antigen 125 (CA125), prostate specific membrane antigen (PSMA), TAG-72 antigen, and fragments thereof.

Various bispecific antibodies comprising a first antibody that specifically binds ErbB3 and a second antibody that specifically binds another protein are described in detail in PCT Publication Nos. WO 2005/117973 and WO 2006/091209 and in U.S. Pat. No. 8,124,085 and U.S. Patent Publication No. 20090246206. Various bispecific antibodies comprising a first antibody that specifically binds ErbB3 and a second antibody that specifically binds another protein, wherein the two antibodies are linked by a modified human serum albumin linker, are described in detail in U.S. Patent Application No. 20110059076 and in PCT Application Nos. WO 2009/126920 and WO 2010/059315.

In yet another embodiment, the anti-ErbB3 agent can comprise two or more anti-ErbB3 antibodies, each of which binds to a different epitope on ErbB3. Preferably, the anti-ErbB3 agent comprises two or three different anti-ErbB3 antibodies, each of which binds to a different epitope on ErbB3.

In yet another embodiment, the anti-ErbB3 agent comprises a soluble ErbB3 receptor, or a soluble ErbB2/ErbB3 receptor complex, capable of binding an ErbB3 ligand (e.g., heregulin). A naturally-occurring soluble secreted form of ErbB3 has been described (p85-soluble ErbB3 or sErbB3. Other soluble forms of ErbB3 have been described as well, see U.S. Pat. No. 7,884,194, and can be prepared by standard recombinant DNA engineering methods through removal of the transmembrane and intracellular domains of ErbB3. In one embodiment, an anti-ErbB3 agent that is a soluble form of ErbB3 comprises a fusion protein, such as an immunoglobulin (Ig) fusion wherein Ig constant domains are linked to the C-terminal end of the soluble form of ErbB3 (ErbB3-Ig fusion protein). The structure and preparation of such Ig fusion proteins are well known in the art (see e.g., U.S. Pat. No. 5,116.).

### III. THERAPEUTIC AGENTS THAT BIND TO AAG

In one embodiment of the disclosed combination therapy methods, a therapeutic agent that binds to alpha 1-acid glycoprotein (AAG) is administered to the patient. AAG is a plasma protein that is known to bind a wide variety of drugs and to be one of the major determinants affecting drug action, distribution and potency. Typically, drugs capable of binding AAG are basic compounds. Accordingly, in one embodiment the AAGB is a basic compound.

In another embodiment, the AAGB is a protein kinase inhibitor. One protein kinase inhibitor that binds to AAG is erlotinib. Other protein tyrosine kinase inhibitors that are known to bind to AAG include imatinib. In another embodiment, the AAGB is an anti-cancer agent. In one embodiment the anti-cancer agent is erlotinib. Non-limiting examples of other anti-cancer agents that are known to bind to AAG include erlotinib, lapatinib, imatinib, gefitinib, nab-paclitaxel and docetaxel.

Non-limiting examples of other drugs that have been demonstrated to bind to AAG include antipsychotic agents such as chloropromazine, haloperidol, risperidone, remoxipride, thioridazine, and carbamazepine; tricyclic anti-depressants such as imipramine, nortriptyline, desipramine, clomipramine, desmethylclomipramine, trimipramine, and amitriptyline; beta blockers such as propanolol and oxprenolol; calcium channel blockers such as verapamil, darodipine, isradipine, nicardipine, and amlodipine; anti-arrhythmic agents such as propafenone, aprindine, and quinidine; as well as acetaminophen; capsaicin; deramciclane; dicumarol; dipyradamole; disopyramide; disopyramide; isoniazid; levosemotiadil; lidocaine (lignocaine); maprotiline; methadone; mifepristone; phenobarbital; phenytoin; progesterone; pyrazinamide; rifampicin; semotiadil; theophylline; valproic acid; vancomycin; and ximelagatran.

In addition, many antiviral agents, e.g., anti-retroviral agents, are known to be AAGBs, these include, e.g., anti-HIV protease inhibitors including ritonavir, indinavir, saquinavir nelfinavir, darunavir, and amprenavir. See, e.g., U.S. Pat. No. 5,750,493.

The ability of a drug to bind to AAG can be determined by any method known in the art, including conventional methods such as equilibrium dialysis and ultrafiltration.

### IV. CANCER TREATMENT

The combination therapy methods disclosed herein are useful for the treatment of cancer. The methods can be used in the treatment of essentially any type of cancer in which targeting ErbB3 would be beneficial (e.g., tumors that express or overexpress ErbB3). Non-limiting examples of types of cancers to be treated include breast cancer, ovarian cancer, renal cancer, gastrointestinal cancer, colon cancer, rectal cancer, colorectal cancer, lung cancer, prostate cancer, prostatic intraepithelial neoplasia, sarcoma, melanoma, head and neck cancer, pancreatic cancer, gall bladder cancer, bladder cancer, cancers of the brain and/or spinal cord, stomach cancer, liver cancer, bone cancer, skin cancer, splenic cancer, testicular cancer, thyroidal cancer, gastric cancer and oral/pharyngeal cancer.

In one embodiment, the cancer is a breast cancer. Examples of types of breast cancers that can be treated include tamoxifen-resistant, estrogen receptor-positive breast cancers, trastuzumab-resistant metastatic breast cancers, hormone-refractory breast cancers and triple negative breast cancers. In another preferred embodiment, the cancer is a colon cancer. In another preferred embodiment, the cancer is a pancreatic cancer. In another embodiment, the cancer is a lung cancer, e.g., a non-small cell lung cancer (NSCLC) or a gefitinib-resistant lung cancer. In another preferred embodiment, the cancer is a sarcoma, preferably a Ewing's sarcoma. In another embodiment, the cancer is bladder cancer. In another embodiment the cancer is a solid tumor. In another embodiment, the cancer is a non-solid tumor, such as a clear cell sarcoma. In some aspects, the cancer is an ErbB2 and ErbB3 positive tumor (e.g., breast tumors and non-small cell lung cancer tumors).

The drugs for combination therapy may be administered to the patient in any suitable form. Typically, a drug is provided in the form of a pharmaceutical composition, which comprises the drug in a physiologically acceptable carrier.

In another aspect, packaged formulations are provided, such as packaged formulations for treating cancers. The packaged formulations can comprise, for example, an anti-ErbB3 agent in a pharmaceutically acceptable carrier and instructions for use in accordance with the combination therapy methods described herein. Preferred anti-ErbB3 agents for use in the packaged formulations are anti-ErbB3 antibodies, such as those described in subsection II above. In another embodiment, a packaged formulation can comprise, for example, an AAGB (e.g., erlotinib) in a pharmaceutically acceptable carrier and instructions for use in accordance with the combination therapy methods described herein.

### V. A RECORDED OR PRINTED WARNING

In another aspect, the methods and packaged formulations are provided for reducing the risk of harmful drug-drug interactions (DDIs) between an anti-ErbB3 agent and a TKI or AAGB, wherein the method or formulation comprises a recorded or printed warning a medical professional with respect to dosages for the anti-ErbB3 agent and/or the TKI or AAGB. For example, in various embodiments, a method is provided for reducing the risk of a harmful DDI, wherein:

the method comprises supplying a tyrosine kinase inhibitor (TKI) to a drug distributor, wherein the TKI is supplied in a container comprising:

a) the TKI formulated for administration to a patient, and

b) a recorded or printed warning a medical professional;

wherein the warning indicates that when the TKI is co-administered to the patient with an ErbB3 inhibitor, a dose reduction of the TKI should be considered; or

the method comprises supplying an ErbB3 inhibitor to a drug distributor, wherein the ErbB3 inhibitor is supplied in a container comprising:

a) the ErbB3 inhibitor formulated for administration to a patient, and

b) a recorded or printed warning a medical professional;

wherein the warning indicates that when the ErbB3 inhibitor is co-administered to the patient with a tyrosine kinase inhibitor (TKI), a dose reduction of the TKI should be considered, or, the warning indicates that when the ErbB3 inhibitor is co-administered to the patient with a TKI, a dose reduction of the ErbB3 inhibitor should be considered; or

the method comprises supplying a drug that is an alpha 1-acid glycoprotein binder (AAGB) to a drug distributor, wherein the AAGB is supplied in a container comprising:

a) the AAGB formulated for administration to a patient, and

b) a recorded or printed warning a medical professional;

wherein the warning indicates that when the AAGB is co-administered to the patient with an ErbB3 inhibitor, a dose reduction of the AAGB should be considered; or

the method comprises supplying an ErbB3 inhibitor to a drug distributor, wherein the ErbB3 inhibitor is supplied in a container comprising:

a) the ErbB3 inhibitor formulated for administration to a patient, and

b) a recorded or printed warning a medical professional;

wherein the warning indicates that when the ErbB3 inhibitor is co-administered to the patient with a drug that is an alpha 1-acid glycoprotein binder (AAGB), a dose reduction of the AAGB should be considered,
or, the warning indicates that when the ErbB3 inhibitor is co-administered to the patient with a drug that is an AAGB, a dose reduction of the ErbB3 inhibitor should be considered.

In another aspect, packaged formulations are provided comprising one or more of an anti-ErbB3 agent, a TKI and/or an AAGB, formulated for administration, and a recorded or printed warning a medical professional with respect to dosages for the anti-ErbB3 agent and/or the TKI or AAGB. For example, various embodiments provide a package, said package comprising a drug in a container, wherein

the drug is a tyrosine kinase inhibitor (TKI) formulated for administration to a patient; the package further comprising a recorded or printed warning a medical professional or the patient, wherein the warning indicates that when the TKI is co-administered with an ErbB3 inhibitor, a dose modification of the TKI should be considered; or

the drug is an ErbB3 inhibitor formulated for administration to a patient; the package further comprising a recorded or printed warning a medical professional or the patient, wherein the warning indicates that when the an ErbB3 inhibitor is co-administered with a tyrosine kinase inhibitor (TKI), a dose modification of the TKI should be considered; or

the drug is an ErbB3 inhibitor formulated for administration to a patient; the package further comprising a recorded or printed warning a medical professional or the patient, wherein the warning indicates that when the an ErbB3 inhibitor is co-administered with a tyrosine kinase inhibitor, a dose modification of the ErbB3 inhibitor should be considered; or

the drug is a tyrosine kinase inhibitor (TKI) formulated for administration to a patient; the package further comprising a recorded or printed warning a medical professional or the patient, wherein the warning indicates that when the TKI is co-administered with an ErbB3 inhibitor, the TKI should be administered at a modified dose; or

the drug is an ErbB3 inhibitor formulated for administration to a patient; the package further comprising a recorded or printed warning a medical professional or the patient, wherein the warning indicates that when the ErbB3 inhibitor is co-administered with a tyrosine kinase inhibitor (TKI), the TKI should be administered at a modified dose; or

the drug is an ErbB3 inhibitor formulated for administration to a patient; the package further comprising a recorded or printed warning a medical professional or the patient, wherein the warning indicates that when the ErbB3 inhibitor is co-administered with a tyrosine kinase inhibitor (TKI), the ErbB3 inhibitor should be administered at a modified dose.

With respect to the above-described methods and packaged formulations, the a recorded or printed warning can comprise, for example, one or more of a recorded audio warning, a recorded video warning, a warning recorded in computer readable form, or a printed warning. A recorded audio and/or video warning may be an apparatus that can play an audio or visual message containing the warning. Various apparatuses incorporating technology for providing such a recorded audio or visual warning are available in the art, such as a smart phone, iPod®, or other digital audio/video player, as well as e.g., those described in U.S. Pat. No. 7,802,386, which are particularly suitable for providing a warning upon the opening of a container. A computer readable form comprising a warning may be, e.g., a magnetic tape, a blue-ray disc, a minidisc, a DVD, a CD-ROM, an external hard drive, a flash drive (e.g. a USB flash drive), or a memory card such as any of the various types of memory cards listed in Table 1, below.

TABLE 1
Memory cards

| Name | Acronym | Name | Acronym |
|---|---|---|---|
| PC Card | PCMCIA | MMCmicro Card | MMCmicro |
| CompactFlash I | CF-I | Secure Digital card | SD |
| CompactFlash II | CF-II | SxS | SxS |
| SmartMedia | SM/SMC | Universal Flash | UFS |
| | | Storage | |
| Memory Stick | MS | miniSD card | miniSD |
| Memory Stick Duo | MSD | microSD card | microSD |
| Memory Stick PRO Duo | MSPD | xD-Picture Card | xD |
| Memory Stick PRO-HG | MSPDX | Intelligent Stick | iStick |
| Duo | | | |
| Memory Stick Micro M2 | M2 | Serial Flash Module | SFM |
| Miniature Card | | μ card | μcard |
| Multimedia Card | MMC | NT Card | NT NT+ |
| Reduced Size | RS-MMC | | |
| Multimedia Card | | | |

### VI. EXAMPLES

The following examples are illustrative and non-limiting.

### Example 1

### Pharmacokinetics of Anti-ErbB3 and Erlotinib Combination Therapy

In this example, human cancer patients were treated with a combination of an anti-ErbB3 monoclonal antibody, MM-121, and a protein kinase inhibitor, erlotinib, and various pharmacokinetic parameters were measured. Patients received one of two different doses of the MM-121 antibody (either 6 mg/kg or 12 mg/kg) administered weekly intravenously. Patients also received one of two different doses of erlotinib (either 100 mg or 150 mg) administered daily orally. The antibody administration began on Day 1 and continued with weekly doses and the erlotinib administration began on Day 2 and continued with daily doses. Pharmacokinetic parameters were monitored for the duration of treatment, until the patient's cancer progressed or the patient came off the study. Each patient received at least two doses of the MM-121 antibody.

For the antibody, the following pharmacokinetic parameters were measured: Tₘₐₓ (estimated time after administration of the antibody to reach maximum concentration in plasma), Cₘₐₓ (maximum concentration of the antibody in plasma observed after administration) and AUC (area under the plasma concentration time curve, as an estimate of bioavailability). For the erlotinib, the average plasma concentration was measured. The results are summarized below in Table 2.

TABLE 2
Pharmacokinetics of MM-121 and Erlotinib Coadministration

| | MM-121 | | | | Erlotinib | |
|---|---|---|---|---|---|---|
| | Dose | Tₘₐₓ | Cₘₐₓ | AUC (0-168) | Dose | Avg Conc. |
| Subject | (mg/kg) | (hrs) | (ng/ml) | hr * ng/ml | (mg) | (ng/ml) |
| 1 | 6 | 1 | 1.74 × 10⁵ | 1.38 × 10⁷ | 100 | 384 |
| 2 | 6 | 1 | 1.13 × 10⁵ | 9.42 × 10⁶ | 150 | 624 |
| 3 | 6 | 1 | 1.18 × 10⁵ | 9.64 × 10⁶ | 150 | 569 |
| 4 | 6 | 1 | 2.30 × 10⁵ | 1.50 × 10⁷ | 150 | 656 |
| 5 | 12 | 1 | 3.51 × 10⁵ | 3.51 × 10⁷ | 150 | 1370 |
| 6 | 12 | 4 | 2.83 × 10⁵ | 2.54 × 10⁷ | 150 | 1165.3 |
| 7 | 12 | 2 | 2.58 × 10⁵ | 1.66 × 10⁷ | 100 | 2395.6 |
| 8 | 12 | 1 | 6.06 × 10⁵ | 3.25 × 10⁷ | 100 | 777.5894 |

The results showed that patients treated with 12 mg/kg of the anti-ErbB3 antibody exhibited an approximately 2-3 fold higher average plasma concentration of erlotinib as compared to patients treated with only 6 mg/kg of the anti-ErbB3 antibody. For example, Patients 2 and 5 were each treated with 150 mg erlotinib but were treated with 6 mg/kg or 12 mg/kg MM-121, respectively, and the average plasma concentration of erlotinib was approximately 2.19 fold higher in Patient 5 than in Patient 2. Similarly, Patients 1 and 8 were each treated with 100 mg erlotinib but were treated with 6 mg/kg or 12 mg/kg MM-121, respectively, and the average plasma concentration of erlotinib was approximately 2.02 fold higher in Patient 8 than in Patient 1.

In view of the foregoing, these results indicate that ErbB3 antagonism in a patient can impact the pharmacokinetics of a companion therapeutic which is a TKI or an AAGB that is being co-administered in combination therapy with the anti-ErbB3 agent. In particular, the results indicate that the average plasma concentration of the co-administered TKI and AAGB drug, such as erlotinib or gefitinib, is increased with increasing amounts of ErbB3 antagonism.

### Example 2

### Combination Dosage Regimens for Anti-ErbB3 and Erlotinib or Gefitinib

A cancer patient in need of treatment with an anti-ErbB3 antibody and erlotinib or gefitinib is selected for treatment. A monotherapy dose for erlotinib treatment is, for example, 150 mg/day. A monotherapy dose for gefitinib treatment is, for example, 250 mg/day. For combination therapy, a reduced dose for erlotinib (as compared to the monotherapy dose of erlotinib of 150 mg/day) or for gefitinib (as compared to the monotherapy dose amount of gefitinib of 250 mg/day) is chosen for co-administration with a monotherapy dose of the antibody (e.g., MM-121 or AMG888). Accordingly, a combination dosage regimen for treatment of the cancer patient is chosen consisting of administration of a monotherapy dose of MM-121 or a monotherapy dose of AMG888 together with (independently) 100 mg/day of erlotinib (a reduced dose as compared to a monotherapy dose for erlotinib) or 125 mg/day of gefitinib (a reduced dose as compared to a monotherapy dose for gefitinib) or 250 mg/kg/48 hrs. (a reduced dose as compared to a monotherapy dose for gefitinib).

### Example 3

### Combination Dosage Regimens for Anti-ErbB3 and Gefitinib or Erlotinib

A cancer patient in need of treatment with an anti-ErbB3 antibody and the tyrosine kinase inhibitor (TKI) gefitinib or the TKI erlotinib is selected for treatment. MM-121 or AMG888 is administered at a monotherapy dose. A monotherapy dose for gefitinib treatment is, for example, 250 mg/day. A monotherapy dose for erlotinib treatment is, for example, 150 mg/day. For combination therapy, a reduced dose of the anti-ErbB3 antibody MM-121 or a reduced dose of the anti-ErbB3 antibody AMG888 is chosen for co-administration with the monotherapy dose of gefitinib or with the monotherapy dose of erlotinib. Accordingly, a combination dosage regimen for treatment of the cancer patient is chosen of: one half of a monotherapy dose of MM-121 or one half of a monotherapy dose of AMG888 together with (independently) 250 mg/day of gefitinib or 150 mg/day of erlotinib.

### Example 4

### Combination Dosage Regimens for Anti-ErbB3 and Erlotinib or Gefitinib

A cancer patient in need of treatment with an anti-ErbB3 antibody and the tyrosine kinase inhibitor (TKI) erlotinib or the TKI gefitinib is selected for treatment. An anti-ErbB3 antibody having a heavy chain amino acid sequence as set forth in SEQ ID NO: 70 of U.S. Pat. No. 7,705,130 and having a light chain amino acid sequence as set forth in SEQ ID NO: 72 of U.S. Pat. No. 7,705,130 is selected for use as the anti-ErbB3 antibody. The anti-ErbB3 antibody is administered to the patient at a concentration of 70 mg/ml and at a monotherapy dose by intravenous infusion once every three weeks. A monotherapy dose for erlotinib is a dose of 150 mg/day. A monotherapy dose for gefitinib is a dose of 250 mg/day. At a monotherapy dose of the anti-ErbB3 antibody, a reduced dosage regimen for erlotinib or a reduced dosage regimen for gefitinib (as compared to the monotherapy dose) is selected and administered to the patient.

### Example 5

### Packaging and Distribution of a Tyrosine Kinase Inhibitor for Combination Therapy

A tyrosine kinase inhibitor (TKI) (e.g., erlotinib or gefitinib) is formulated for administration to a patient, put into a container and then packaged into a package, wherein the package also includes a warning, such as a recorded audio warning, a recorded video warning, a warning recorded in computer readable form or a printed warning, for a medical professional (e.g., a physician). This warning indicates that when the TKI is co-administered to a patient with an ErbB3 inhibitor (e.g., an anti-ErbB3 antibody), a dose modification of the TKI, such as a dose reduction of the TKI, should be considered. The warning further indicates that a dose reduction in, for example, 25 mg or 50 mg or about 62 mg or 125 mg increments per dose of the TKI is suggested. The package, comprising the TKI inhibitor formulation in a container and the warning for the medical professional, is supplied to a drug distributor.

### Example 6

### Formulation and Distribution of an Anti-ErbB3 Antibody for Combination Therapy

An anti-ErbB3 antibody, e.g., MM-121 or AMG888, is formulated for administration to a patient, put into a container and then packaged into a package, wherein the package also includes a warning, such as a recorded audio warning, a recorded video warning, a warning recorded in computer readable form or a printed warning, for a medical professional. This warning indicates that when the anti-ErbB3 antibody is co-administered to a patient with a tyrosine kinase inhibitor (TKI) (e.g., erlotinib or gefitinib), a dose modification of the TKI, such as a dose reduction of the TKI, should be considered. The warning optionally further indicates that a dose reduction in, for example, 25 mg or 50 mg or about 62 mg or 125 mg increments per dose of the TKI is suggested. The package, comprising the anti-ErbB3 antibody formulation in a container and the warning for the medical professional, is supplied to a drug distributor.

SUMMARY OF SEQUENCES

```
MM-121 V_{H} amino acid sequence (SEQ ID NO: 1)
EVQLLESGGGLVQPGGSLRLSCAASGFTFSHYVMAWVRQAPGKGLEWVSSIS
SSGGWTLYADSVKGRFTISRDNSKNTLYLQMNSLRAEDTAVYYCTRGLKMATIFDY
WGQGTLVTVSS
MM-121 V_{L} amino acid sequence (SEQ ID NO: 2)
QSALTQPASVSGSPGQSITISCTGTSSDVGSYNVVSWYQQHPGKAPKLIIYEVS
QRPSGVSNRFSGSKSGNTASLTISGLQTEDEADYYCCSYAGSSIFVIFGGGTKVTVL
MM-121 V_{H} CDR1 (SEQ ID NO: 3)
HYVMA
MM-121 V_{H} CDR2 (SEQ ID NO: 4)
SISSSGGWTLYADSVKG
MM-121 V_{H} CDR3 (SEQ ID NO: 5)
GLKMATIFDY
MM-121 V_{L} CDR1 (SEQ ID NO: 6)
TGTSSDVGSYNVVS
MM-121 V_{L} CDR2 (SEQ ID NO: 7)
EVSQRPS
MM-121 V_{L} CDR3 (SEQ ID NO: 8)
CSYAGSSIFVI
MM-121 heavy chain amino acid sequence (SEQ ID NO: 9)
    1 EVQLLESGGG LVQPGGSLRL SCAASGFTFS HYVMAWVRQA PGKGLEWVSS
  51 ISSSGGWTLY ADSVKGRFTI SRDNSKNTLY LQMNSLRAED TAVYYCTRGL
101 KMATIFDYWG QGTLVTVSSA STKGPSVFPL APCSRSTSES TAALGCLVKD
151 YFPEPVTVSW NSGALTSGVH TFPAVLQSSG LYSLSSVVTV PSSNFGTQTY
201 TCNVDHKPSN TKVDKTVERK CCVECPPCPA PPVAGPSVFL FPPKPKDTLM
251 ISRTPEVTCV VVDVSHEDPE VQFNWYVDGV EVHNAKTKPR EEQFNSTFRV
301 VSVLTVVHQD WLNGKEYKCK VSNKGLPAPI EKTISKTKGQ PREPQVYTLP
351 PSREEMTKNQ VSLTCLVKGF YPSDIAVEWE SNGQPENNYK TTPPMLDSDG
401 SFFLYSKLTV DKSRWQQGNV FSCSVMHEAL HNHYTQKSLS LSPGK
MM-121 light chain amino acid sequence (SEQ ID NO: 10)
    1 QSALTQPASV SGSPGQSITI SCTGTSSDVG SYNVVSWYQQ HPGKAPKLII
  51 YEVSQRPSGV SNRFSGSKSG NTASLTISGL QTEDEADYYC CSYAGSSIFV
101 IFGGGTKVTV LGQPKAAPSV TLFPPSSEEL QANKATLVCL VSDFYPGAVT
151 VAWKADGSPV KVGVETTKPS KQSNNKYAAS SYLSLTPEQW KSHRSYSCRV
201 THEGSTVEKT VAPAECS
Ab # 3 V_{H} amino acid sequence (SEQ ID NO: 11)
EVQLLESGGGLVQPGGSLRLSCAASGFTFSAYNMRWVRQAPGKGLEWVSVI
YPSGGATRYADSVKGRFTISRDNSKNTLYLQMNSLRAEDTAVYYCARGYYYYGMD
VWGQGTLVTVSS
Ab # 3 V_{L} amino acid sequence (SEQ ID NO: 12)
QSVLTQPPSASGTPGQRVTISCSGSDSNIGRNYIYWYQQFPGTAPKLLIYRNNQ
RPSGVPDRISGSKSGTSASLAISGLRSEDEAEYHCGTWDDSLSGPVFGGGTKLTVL
Ab # 3 V_{H} CDR1 (SEQ ID NO: 13)
AYNMR
Ab # 3 V_{H} CDR2 (SEQ ID NO: 14)
VIYPSGGATRYADSVKG
Ab # 3 V_{H} CDR3 (SEQ ID NO: 15)
GYYYYGMDV
Ab # 3 V_{L} CDR1 (SEQ ID NO: 16)
SGSDSNIGRNYIY
Ab # 3 V_{L} CDR2 (SEQ ID NO: 17)
RNNQRPS
Ab # 3 V_{L} CDR3 (SEQ ID NO: 18)
GTWDDSLSGPV
Ab # 14 V_{H} amino acid sequence (SEQ ID NO: 19)
EVQLLESGGGLVQPGGSLRLSCAASGFTFSAYGMGWVRQAPGKGLEWVSYI
SPSGGHTKYADSVKGRFTISRDNSKNTLYLQMNSLRAEDTAVYYCAKVLETGLLVD
AFDIWGQGTMVTVSS
Ab # 14 V_{L} amino acid sequence (SEQ ID NO: 20)
QYELTQPPSVSVYPGQTASITCSGDQLGSKFVSWYQQRPGQSPVLVMYKDKR
RPSEIPERFSGSNSGNTATLTISGTQAIDEADYYCQAWDSSTYVFGTGTKVTVL
Ab # 14 V_{H} CDR1 (SEQ ID NO: 21)
AYGMG
Ab # 14 V_{H} CDR2 (SEQ ID NO: 22)
YISPSGGHTKYADSVKG
Ab # 14 V_{H} CDR3 (SEQ ID NO: 23)
VLETGLLVDAFDI
Ab # 14 V_{L} CDR1 (SEQ ID NO: 24)
SGDQLGSKFVS
Ab # 14 V_{L} CDR2 (SEQ ID NO: 25)
YKDKRRPS
Ab # 14 V_{L} CDR3 (SEQ ID NO: 26)
QAWDSSTYV
Ab # 17 V_{H} amino acid sequence (SEQ ID NO: 27)
EVQLLESGGGLVQPGGSLRLSCAASGFTFSWYGMGWVRQAPGKGLEWVSYI
SPSGGITVYADSVKGRFTISRDNSKNTLYLQMNSLRAEDTAVYYCARLNYYYGLDV
WGQGTTVTVSS
Ab # 17 V_{L} amino acid sequence (SEQ ID NO: 28)
QDIQMTQSPSSLSASVGDRITITCQASQDIGDSLNWYQQKPGKAPRLLIYDAS
NLETGVPPRFSGSGSGTDFTFTFRSLQPEDIATYFCQQSANAPFTFGPGTKVDIK
Ab # 17 V_{H} CDR1 (SEQ ID NO: 29)
WYGMG
Ab # 17 V_{H} CDR2 (SEQ ID NO: 30)
YISPSGGITVYADSVKG
Ab # 17 V_{H} CDR3 (SEQ ID NO: 31)
LNYYYGLDV
Ab # 17 V_{L} CDR1 (SEQ ID NO: 32)
QASQDIGDSLN
Ab # 17 V_{L} CDR2 (SEQ ID NO: 33)
DASNLET
Ab # 17 V_{L} CDR3 (SEQ ID NO: 34)
QQSANAPFT
Ab # 19 V_{H} amino acid sequence (SEQ ID NO: 35)
EVQLLESGGGLVQPGGSLRLSCAASGFTFSRYGMWWVRQAPGKGLEWVSYI
GSSGGPTYYVDSVKGRFTISRDNSKNTLYLQMNSLRAEDTAVYYCAGGRGTPYYFD
SWGQGTLVTVSS
Ab # 19 V_{L} amino acid sequence (SEQ ID NO: 36)
QYELTQPASVSGSPGQSITISCTGTSSDIGRWNIVSWYQQHPGKAPKLMIYDV
SNRPSGVSNRFSGSKSGNTASLTISGLQAEDEADYYCSSYTSSSTWVFGGGTKLTVL
Ab # 19 V_{H} CrR1 (SEQ ID NO: 37)
RYGMW
Ab # 19 V_{H} CDR2 (SEQ ID NO: 38)
YIGSSGGPTYYVDSVKG
Ab # 19 V_{H} CDR3 (SEQ ID NO: 39)
GRGTPYYFDS
Ab # 19 V_{L} CDR1 (SEQ ID NO: 40)
TGTSSDIGRWNIVS
Ab # 19 V_{L} CDR2 (SEQ ID NO: 41)
DVSNRPS
Ab # 19 V_{L} CDR3 (SEQ ID NO: 42)
SSYTSSSTWV
ErbB3 (SEQ ID NO: 43)
SEVGNSQAVCPGTLNGLSVTGDAENQYQTLYKLYERCEVVMGNLEIVLTGHNADLS
FLQWIREVTGYVLVAMNEFSTLPLPNLRVVRGTQVYDGKFAIFVMLNYNTNSSHAL
RQLRLTQLTEILSGGVYIEKNDKLCHMDTIDWRDIVRDRDAEIVVKDNGRSCPPCHE
VCKGRCWGPGSEDCQTLTKTICAPQCNGHCFGPNPNQCCHDECAGGCSGPQDTDCF
ACRHFNDSGACVPRCPQPLVYNKLTFQLEPNPHTKYQYGGVCVASCPHNFVVDQTS
CVRACPPDKMEVDKNGLKMCEPCGGLCPKACEGTGSGSRFQTVDSSNIDGFVNCTK
ILGNLDFLITGLNGDPWHKIPALDPEKLNVFRTVREITGYLNIQSWPPHMHNFSVFSN
LTTIGGRSLYNRGFSLLIMKNLNVTSLGFRSLKEISAGRIYISANRQLCYHHSLNWTK
VLRGPTEERLDIKHNRPRRDCVAEGKVCDPLCSSGGCWGPGPGQCLSCRNYSRGGV
CVTHCNFLNGEPREFAHEAECFSCHPECQPMEGTATCNGSGSDTCAQCAHFRDGPH
CVSSCPHGVLGAKGPIYKYPDVQNECRPCHENCTQGCKGPELQDCLGQTLVLIGKTH
LTMALTVIAGLVVIFMMLGGTFLYWRGRRIQNKRAMRRYLERGESIEPLDPSEKANK
VLARIFKETELRKLKVLGSGVFGTVHKGVWIPEGESIKIPVCIKVIEDKSGRQSFQAVT
DHMLAIGSLDHAHIVRLLGLCPGSSLQLVTQYLPLGSLLDHVRQHRGALGPQLLLN
WGVQIAKGMYYLEEHGMVHRNLAARNVLLKSPSQVQVADFGVADLLPPDDKQLL
YSEAKTPIKWMALESIHFGKYTHQSDVWSYGVTVWELMTFGAEPYAGLRLAEVPDL
LEKGERLAQPQICTIDVYMVMVKCWMIDENIRPTFKELANEFTRMARDPPRYLVIKR
ESGPGIAPGPEPHGLTNKKLEEVELEPELDLDLDLEAEEDNLATTTLGSALSLPVGTL
NRPRGSQSLLSPSSGYMPMNQGNLGESCQESAVSGSSERCPRPVSLHPMPRGCLASE
SSEGHVTGSEAELQEKVSMCRSRSRSRSPRPRGDSAYHSQRHSLLTPVTPLSPPGLEE
EDVNGYVMPDTHLKGTPSSREGTLSSVGLSSVLGTEEEDEDEEYEYMNRRRRHSPPH
PPRPSSLEELGYEYMDVGSDLSASLGSTQSCPLHPVPIMPTAGTTPDEDYEYMNRQR
DGGGPGGDYAAMGACPASEQGYEEMRAFQGPGHQAPHVHYARLKTLRSLEATDSA
FDNPDYWHSRLFPKANAQRT
```

### VII. INCORPORATION BY REFERENCE

The disclosure of each and every US, International, or other patent or patent application or publication referred to herein is hereby incorporated herein by reference in its entirety.

1. A method for reducing the risk of a harmful drug-drug interaction, the method comprising supplying a tyrosine kinase inhibitor (TKI) to a drug distributor, wherein the TKI is supplied in a container comprising:
a) the TKI formulated for administration to a patient, and
b) a recorded or printed warning to a medical professional;
wherein the warning indicates that when the TKI is co-administered to the patient with an ErbB3 inhibitor, a dose reduction of the TKI should be considered.

2. A method for reducing the risk of a harmful drug-drug interaction, the method comprising supplying an ErbB3 inhibitor to a drug distributor, wherein the ErbB3 inhibitor is supplied in a container comprising:
a) the ErbB3 inhibitor formulated for administration to a patient, and
b) a recorded or printed warning to a medical professional;
wherein the warning indicates that:
(a) when the ErbB3 inhibitor is co-administered to the patient with a tyrosine kinase inhibitor (TKI), a dose reduction of the TKI or ErbB3 inhibitor should be considered; or
(b) when the ErbB3 inhibitor is co-administered to the patient with a drug that is an alpha 1-acid glycoprotein binder (AAGB), a dose reduction of the ErbB3 inhibitor or AAGB should be considered.

3. A method for reducing the risk of a harmful drug-drug interaction, the method comprising supplying a drug that is an alpha 1-acid glycoprotein binder (AAGB) to a drug distributor, wherein the AAGB is supplied in a container comprising:
a) the AAGB formulated for administration to a patient, and
b) a recorded or printed warning to a medical professional;
wherein the warning indicates that when the AAGB is co-administered to the patient with an ErbB3 inhibitor, a dose reduction of the AAGB should be considered.

4. (canceled)

5. The method of claim 1, wherein the recorded or printed warning comprises one or more of a recorded audio warning, a recorded video warning, a warning recorded in computer readable form, or a printed warning.

6. The method of claim 1, wherein the medical professional is a physician, a physician's assistant, a nurse, or a pharmacist.

7. The method of claim 1, wherein:
(a) the TKI is erlotinib and the warning further indicates that a dosage reduction in 25 mg or 50 mg increments is suggested; or
(b) the TKI is gefitinib and the warning further indicates that a dosage reduction in 125 or about 62 mg increments is suggested.

8. (canceled)

9. The method of claim 1, wherein the patient is suffering from a cancer.

10. (canceled)

11. The method of claim 1, wherein the ErbB3 inhibitor is an anti-ErbB3 antibody.

12. The method of claim 11 wherein the anti-ErbB3 antibody is an antibody having the heavy chain sequence set forth in SEQ ID NO:1 and the light chain sequence set forth in SEQ ID NO:2.

13. A package, said package comprising a drug in a container, wherein the drug is a tyrosine kinase inhibitor (TKI) formulated for administration to a patient; the package further comprising a recorded or printed warning to a medical professional or the patient, wherein the warning indicates that when the TKI is co-administered with an ErbB3 inhibitor, a dose modification of the TKI should be considered.

14. A package, said package comprising a drug in a container, wherein the drug is an ErbB3 inhibitor formulated for administration to a patient; the package further comprising a recorded or printed warning to a medical professional or the patient, wherein the warning indicates that when the ErbB3 inhibitor is co-administered with a tyrosine kinase inhibitor (TKI), a dose modification of the ErbB3 inhibitor or TKI should be considered.

15. (canceled)

16. The package of claim 13, wherein the dose modification is a dose reduction.

17. (canceled)

18. (canceled)

19. (canceled)

20. (canceled)

21. A method of combination therapy, the therapy being for treatment of a patient suffering from a cancer, the combination therapy being administration of both of an anti-ErbB3 drug and either a tyrosine kinase inhibitor (TKI) drug or an alpha 1 acid glycoprotein binding (AAGB) drug, the method comprising administering both the anti-ErbB3 drug and the TKI drug or the AAGB drug to the patient, wherein either or both of 1) the TKI drug or the AAGB drug, and 2) the anti-ErbB3 drug, is administered to the patient in one or more modified doses as compared to the monotherapy dose treatment with the drug in such a patient.

22. (canceled)

23. The method of claim 21, wherein:
(a) the TKI drug or the AAGB drug is administered at the reduced dose and the anti-ErbB3 drug is not administered at the reduced dose;
(b) the anti-ErbB3 drug is administered at the reduced dose and the TKI drug or the AAGB drug is not administered at the reduced dose; or
(c) each of 1) the TKI drug or the AAGB drug, and 2) the anti-ErbB3 drug, are administered at a reduced dose.

24. (canceled)

25. (canceled)

26. The method of claim 23, wherein the amount of each reduced dose is reduced by 10-75%.

27. (canceled)

28. (canceled)

29. (canceled)

30. (canceled)

31. (canceled)

32. (canceled)

33. The method of claim 21, wherein
the anti-ErbB3 drug exhibits a first serum half life;
the TKI drug or the AAGB drug exhibits a second serum half life; and
administration of the anti-ErbB3 drug to the patient occurs within 1 to 3 first serum half lives before administering the TKI drug or the AAGB drug; or
administration of the anti-ErbB3 drug occurs within 1 to 3 second serum half lives after administering the TKI drug or the AAGB drug.

34. The method of claim 21, wherein the anti-ErbB3 drug is an anti-ErbB3 antibody.

35. The method of claim 34, wherein the anti-ErbB3 antibody is an antibody having the heavy chain sequence set forth in SEQ ID NO:1 and the light chain sequence set forth in SEQ ID NO:2.

36. The method of claim 33, wherein the anti-ErbB3 drug is a bispecific antibody comprising an anti-ErbB3 antibody linked to a second antibody.

37. The method of claim 21, wherein the AAGB drug is a basic compound or an anti-cancer agent.

38. (canceled)

39. The method of claim 21, wherein the TKI drug is erlotinib or gefitinib.

40. The method of claim 34, wherein the anti-ErbB3 antibody is administered to the patient in a modified dose and wherein the modified dose is a reduced dose of about ½ or about ¼ of the monotherapy dose with the anti-ErbB3 antibody.

41. The method of claim 39, wherein:
(a) the monotherapy dose for erlotinib is 150 mg/day and the reduced dose for erlotinib is 125 mg/day;
(b) the monotherapy dose for erlotinib is 150 mg/day and reduced dose for erlotinib is 75 mg/day or 50 mg/day;
(c) the monotherapy dose for erlotinib is 100 mg/day and the reduced dose for erlotinib is 75 mg/day or 50 mg/day or 25 mg/day; or
(d) the monotherapy dose for erlotinib is 150 mg/day and the reduced dose for erlotinib is 100 mg/day.

42. (canceled)

43. (canceled)

44. (canceled)

45. The method of claim 39, wherein the monotherapy dose for gefitinib is 250 mg/day and the reduced dose for gefitinib is 150 mg/day or 125 mg/day or 100 mg/day; or about 62 mg/day, or 50 mg/day.

46. The method of claim 21, wherein the cancer is selected from the group consisting of breast cancer, ovarian cancer, renal cancer, gastrointestinal cancer, colon cancer, rectal cancer, colorectal cancer, lung cancer, prostate cancer, prostatic intraepithelial neoplasia, sarcoma, melanoma, head and neck cancer, pancreatic cancer, gall bladder cancer, bladder cancer, cancers of the brain and/or spinal cord, stomach cancer, liver cancer, bone cancer, skin cancer, splenic cancer, testicular cancer, thyroidal cancer, gastric cancer and oral/pharyngeal cancer.

47. A packaged formulation for treating cancer comprising an anti-ErbB3 drug in a pharmaceutically acceptable carrier and instructions for use in accordance with the method of claim 21.

48. A packaged formulation for treating cancer comprising an anti-ErbB3 antibody in a pharmaceutically acceptable carrier and instructions for use in accordance with the method of claim 21.

49. The method of claim 21, wherein the patient is a cigarette smoker, and wherein the patient is given a modified dose of a TKI drug that is higher than the modified dose of the TKI drug that would be given to the same patient if the same patient were a non-smoker.

50. The method of claim 21, wherein the patient is a smoker, and the dose of the anti-ErbB3 drug is higher than the modified dose of the anti-ErbB3 drug that would be given to the same patient if the same patient were a non-smoker.

51. The package of claim 13, wherein the modification is a reduction and the warning indicates that the reduction for a patient who is a cigarette smoker should be a reduction of a lesser magnitude than the reduction for a patient who is not a cigarette smoker.


SEQUENCE LISTING (43 sequences)

SEQ ID NO: 1 - protein, 119 aa, Artificial Sequence [sequence omitted]

SEQ ID NO: 2 - protein, 111 aa, Artificial Sequence [sequence omitted]

SEQ ID NO: 3 - protein, 5 aa, Artificial Sequence
  Description of Artificial Sequence Synthetic peptide
HYVMA

SEQ ID NO: 4 - protein, 17 aa, Artificial Sequence
  Description of Artificial Sequence Synthetic peptide
SISSSGGWTLYADSVKG

SEQ ID NO: 5 - protein, 10 aa, Artificial Sequence
  Description of Artificial Sequence Synthetic peptide
GLKMATIFDY

SEQ ID NO: 6 - protein, 14 aa, Artificial Sequence
  Description of Artificial Sequence Synthetic peptide
TGTSSDVGSYNVVS

SEQ ID NO: 7 - protein, 7 aa, Artificial Sequence
  Description of Artificial Sequence Synthetic peptide
EVSQRPS

SEQ ID NO: 8 - protein, 11 aa, Artificial Sequence
  Description of Artificial Sequence Synthetic peptide
CSYAGSSIFVI

SEQ ID NO: 9 - protein, 445 aa, Artificial Sequence
  Description of Artificial Sequence Synthetic polypeptide
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 217 aa, Artificial Sequence
  Description of Artificial Sequence Synthetic polypeptide
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 11 - protein, 118 aa, Artificial Sequence
  Description of Artificial Sequence Synthetic polypeptide
EVQLLESGGGLVQPGGSLRLSCAASGFTFSAYNMRWVRQAPGKGLEWVSVIYPSGGATRYADSVKGRFTISRDNSKNTLYLQMNSLRAEDTAVYYCARGYYYYGMDVWGQGTLVTVSS

SEQ ID NO: 12 - protein, 110 aa, Artificial Sequence
  Description of Artificial Sequence Synthetic polypeptide
QSVLTQPPSASGTPGQRVTISCSGSDSNIGRNYIYWYQQFPGTAPKLLIYRNNQRPSGVPDRISGSKSGTSASLAISGLRSEDEAEYHCGTWDDSLSGPVFGGGTKLTVL

SEQ ID NO: 13 - protein, 5 aa, Artificial Sequence
  Description of Artificial Sequence Synthetic peptide
AYNMR

SEQ ID NO: 14 - protein, 17 aa, Artificial Sequence
  Description of Artificial Sequence Synthetic peptide
VIYPSGGATRYADSVKG

SEQ ID NO: 15 - protein, 9 aa, Artificial Sequence
  Description of Artificial Sequence Synthetic peptide
GYYYYGMDV

SEQ ID NO: 16 - protein, 13 aa, Artificial Sequence
  Description of Artificial Sequence Synthetic peptide
SGSDSNIGRNYIY

SEQ ID NO: 17 - protein, 7 aa, Artificial Sequence
  Description of Artificial Sequence Synthetic peptide
RNNQRPS

SEQ ID NO: 18 - protein, 11 aa, Artificial Sequence
  Description of Artificial Sequence Synthetic peptide
GTWDDSLSGPV

SEQ ID NO: 19 - protein, 122 aa, Artificial Sequence
  Description of Artificial Sequence Synthetic polypeptide
EVQLLESGGGLVQPGGSLRLSCAASGFTFSAYGMGWVRQAPGKGLEWVSYISPSGGHTKYADSVKGRFTISRDNSKNTLYLQMNSLRAEDTAVYYCAKVLETGLLVDAFDIWGQGTMVTVSS

SEQ ID NO: 20 - protein, 106 aa, Artificial Sequence
  Description of Artificial Sequence Synthetic polypeptide
QYELTQPPSVSVYPGQTASITCSGDQLGSKFVSWYQQRPGQSPVLVMYKDKRRPSEIPERFSGSNSGNTATLTISGTQAIDEADYYCQAWDSSTYVFGTGTKVTVL

SEQ ID NO: 21 - protein, 5 aa, Artificial Sequence
  Description of Artificial Sequence Synthetic peptide
AYGMG

SEQ ID NO: 22 - protein, 17 aa, Artificial Sequence
  Description of Artificial Sequence Synthetic peptide
YISPSGGHTKYADSVKG

SEQ ID NO: 23 - protein, 13 aa, Artificial Sequence
  Description of Artificial Sequence Synthetic peptide
VLETGLLVDAFDI

SEQ ID NO: 24 - protein, 11 aa, Artificial Sequence
  Description of Artificial Sequence Synthetic peptide
SGDQLGSKFVS

SEQ ID NO: 25 - protein, 8 aa, Artificial Sequence
  Description of Artificial Sequence Synthetic peptide
YKDKRRPS

SEQ ID NO: 26 - protein, 9 aa, Artificial Sequence
  Description of Artificial Sequence Synthetic peptide
QAWDSSTYV

SEQ ID NO: 27 - protein, 118 aa, Artificial Sequence
  Description of Artificial Sequence Synthetic polypeptide
EVQLLESGGGLVQPGGSLRLSCAASGFTFSWYGMGWVRQAPGKGLEWVSYISPSGGITVYADSVKGRFTISRDNSKNTLYLQMNSLRAEDTAVYYCARLNYYYGLDVWGQGTTVTVSS

SEQ ID NO: 28 - protein, 108 aa, Artificial Sequence
  Description of Artificial Sequence Synthetic polypeptide
QDIQMTQSPSSLSASVGDRITITCQASQDIGDSLNWYQQKPGKAPRLLIYDASNLETGVPPRFSGSGSGTDFTFTFRSLQPEDIATYFCQQSANAPFTFGPGTKVDIK

SEQ ID NO: 29 - protein, 5 aa, Artificial Sequence
  Description of Artificial Sequence Synthetic peptide
WYGMG

SEQ ID NO: 30 - protein, 17 aa, Artificial Sequence
  Description of Artificial Sequence Synthetic peptide
YISPSGGITVYADSVKG

SEQ ID NO: 31 - protein, 9 aa, Artificial Sequence
  Description of Artificial Sequence Synthetic peptide
LNYYYGLDV

SEQ ID NO: 32 - protein, 11 aa, Artificial Sequence
  Description of Artificial Sequence Synthetic peptide
QASQDIGDSLN

SEQ ID NO: 33 - protein, 7 aa, Artificial Sequence
  Description of Artificial Sequence Synthetic peptide
DASNLET

SEQ ID NO: 34 - protein, 9 aa, Artificial Sequence
  Description of Artificial Sequence Synthetic peptide
QQSANAPFT

SEQ ID NO: 35 - protein, 119 aa, Artificial Sequence
  Description of Artificial Sequence Synthetic polypeptide
EVQLLESGGGLVQPGGSLRLSCAASGFTFSRYGMWWVRQAPGKGLEWVSYIGSSGGPTYYVDSVKGRFTISRDNSKNTLYLQMNSLRAEDTAVYYCAGGRGTPYYFDSWGQGTLVTVSS

SEQ ID NO: 36 - protein, 110 aa, Artificial Sequence
  Description of Artificial Sequence Synthetic polypeptide
QYELTQPASVSGSPGQSITISCTGTSSDIGRWNIVSWYQQHPGKAPKLMIYDVSNRPSGVSNRFSGSKSGNTASLTISGLQAEDEADYYCSSYTSSSTWVFGGGTKLTVL

SEQ ID NO: 37 - protein, 5 aa, Artificial Sequence
  Description of Artificial Sequence Synthetic peptide
RYGMW

SEQ ID NO: 38 - protein, 17 aa, Artificial Sequence
  Description of Artificial Sequence Synthetic peptide
YIGSSGGPTYYVDSVKG

SEQ ID NO: 39 - protein, 10 aa, Artificial Sequence
  Description of Artificial Sequence Synthetic peptide
GRGTPYYFDS

SEQ ID NO: 40 - protein, 14 aa, Artificial Sequence
  Description of Artificial Sequence Synthetic peptide
TGTSSDIGRWNIVS

SEQ ID NO: 41 - protein, 7 aa, Artificial Sequence
  Description of Artificial Sequence Synthetic peptide
DVSNRPS

SEQ ID NO: 42 - protein, 10 aa, Artificial Sequence
  Description of Artificial Sequence Synthetic peptide
SSYTSSSTWV

SEQ ID NO: 43 - protein, 1323 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT